(19) United States
(12) Patent Application Publication
(10) Pub. No.: US 2025/0132042 A1
(43) Pub. Date: Apr. 24, 2025

(54) PHYSIOLOGICAL BIOMARKER-RELATED MULTIMODAL DATA COLLECTION

(71) Applicant: DISNEY ENTERPRISES, INC., Burbank, CA (US)

(72) Inventors: Keith J. Comito, Seaford, NY (US); Gregory Brooks Hale, Orlando, FL (US); Gary David Markowitz, San Juan Capistrano, CA (US)

(21) Appl. No.: 18/381,687

(22) Filed: Oct. 19, 2023

(51) Int. Cl. G16H 50/30; A61B 5/00

(52) U.S. Cl. CPC G16H 50/30; A61B 5/7264

(57) ABSTRACT

A method is disclosed that includes collecting first biomarker-related data of a subject from a first sensor and second biomarker-related of the subject from a second sensor, the first biomarker-related data being tagged with a first time of collection of the first biomarker-related data from the first sensor, the second biomarker-related data being tagged with a second time of collection of the second biomarker-related data from the second sensor; generating a first physiological biomarker using the first biomarker-related data and a second physiological biomarker using the second biomarker-related data; and determining a health status of the subject based on the first physiological biomarker and the second physiological biomarker.

### FIELD

The present application relates to systems and methods of collecting physiological biomarker related data from subjects and use of such data to predict or determine health status of the subjects.

### BACKGROUND

Various physiological biometrics (such as gait related metrics, vocal metrics, and the like) may be predictive of overall health condition and/or presence of specific health issues or diseases. For example, gait related metrics and vocal metrics may be predictive of heat stress. However, collection of biometric data used to generate such physiological biometrics is generally time consuming and cumbersome, such that it is difficult to utilize the physiological biometrics for detecting or determining overall health conditions in real-time or near real-time.

### BRIEF SUMMARY

In one embodiment, a method is disclosed that includes collecting first biomarker-related data of a subject from a first sensor and second biomarker-related of the subject from a second sensor, the first biomarker-related data being tagged with a first time of collection of the first biomarker-related data from the first sensor, the second biomarker-related data being tagged with a second time of collection of the second biomarker-related data from the second sensor; generating a first physiological biomarker using the first biomarker-related data and a second physiological biomarker using the second biomarker-related data; and determining a health status of the subject based on the first physiological biomarker and the second physiological biomarker.

In another example, one or more non-transitory computer readable media encoded with instructions are disclosed where the instructions when executed cause the processors to receive a plurality of receive a plurality of physiological biomarkers generated based on biomarker-related data collected from a plurality of subjects, the plurality of physiological biomarkers being associated with a plurality of health statuses of the plurality of subjects; identify at least a first physiological biomarker of the plurality of physiological biomarkers and a second physiological biomarker of the plurality of physiological biomarkers associated with a health status of the plurality of health statuses; and generate a machine learning model configured to determine the health status based on input related to the first physiological biomarker and the second physiological biomarker.

In yet another embodiment, a biomarker correlation system is disclosed that includes a plurality of sensors configured to collect biomarker related data from a subject and one or more processors. The processors are configured to receive first biomarker related data of the subject from a first sensor of the plurality of sensors and second biomarker related data of the subject from a second sensor of the plurality of sensors, generate a first physiological biomarker using the first biomarker related data and a second physiological biomarker using the second biomarker related data, and determine a health status of the subject based of the first physiological biomarker and the second physiological biomarker.

### BRIEF DESCRIPTION OF THE SEVERAL VIEWS OF THE DRAWINGS

FIG. 1A illustrates an example multimodal biomarker analysis system in use by a subject.

FIG. 1B illustrates an additional example multimodal biomarker analysis system in use by a subject.

FIG. 2A illustrates a schematic diagram of an example multimodal data collection unit.

FIG. 2B illustrates a schematic diagram of an additional example multimodal data collection unit.

FIG. 3 illustrates a schematic diagram of an example biomarker analysis system.

FIG. 4 is an example computing system used in various examples of the disclosure.

FIG. 5 is a flow chart illustrating an example process for determining a health status of a subject using correlations between physiological biomarkers.

FIG. 6 is a flow chart illustrating an example process for generating machine learning models for detecting health status using physiological biomarkers and cross-correlations of physiological biomarkers.

### DETAILED DESCRIPTION

Physiological biometrics based on non-invasive data collection have been shown to be highly predictive in determining an overall health state or specific condition of an analyzed subject. For example, walking and gait related metrics obtained from video data, vocal quality or speech metrics derived from audio data, and other such biomarkers are predictive of various conditions or health states such as overall health, mental or emotional state, heat stress, disease presence, disease susceptibility, rate of biological aging, and the like. The predictive value of these physiological biomarkers may increase with concurrent collection of multiple types of data used to generate the physiological biomarkers and analysis of multiple physiological biomarkers together. For example, a subject experiencing gait irregularities at the same time as vocal irregularities may be likely experiencing heat stress. In this example, a system able to collect video data and vocal data concurrently and to generate and analyze gait and vocal biomarkers may be useful in detecting heat stress in real-time.

The systems and methods described herein may utilize multimodal data collection to improve predictions of various health statuses. As used herein, multimodal data collection may include collection of multiple types of data from multiple sensors, such as, for example, thermal imaging data, audio data streams, video data streams, tactile input from a subject, and the like. Collection of such multimodal data creates a more robust data set, and generally increases predictive value of the data. The different types of data may be cross-referenced to one another, such as through time alignment (e.g., using time stamps), creating the robust data set which may be utilized for training of machine learning models, determination of health status, and the like.

The systems and methods described herein may be utilized to determine health status of a subject based on physiological biomarkers derived from biomarker-related data. Biomarker-related data may be, for example, any type of data from which physiological biomarkers can be derived. For example, walking-gait related biomarkers may be derived from video data. In one example, systems described herein may be utilized to identify heat exhaustion from multiple types of collected biomarker-related data. For example, a multimodal data collection unit may include a camera collecting video of a subject, microphones collecting vocal recordings of the subject, and a thermal camera collecting thermal imaging of the subject. The collected video may be utilized to identify characteristics of the subject's gait (e.g., speed, cadence, symmetry, and the like). Vocal recordings may be utilized to identify breathing patterns of the subject, characteristics of the subject's speech patterns, vocal cord related biomarkers, cognitive measurements, and the like. Similarly, thermal imaging may be utilized to determine the subject's body temperature. Such physiological biomarkers may be time aligned to predict whether the subject is at risk for, or currently experiencing, heat exhaustion.

In various examples, the biomarker-related data and/or the physiological biomarkers may be associated with timestamps indicating the time at which the initial biomarker-related data was collected from the subject. For example, video data and audio data may include timestamps indicated when the frames of the audio and video data were recorded. Similarly, other data may be associated with timestamps indicating when the data was collected. The various physiological biomarkers may be correlated using these timestamps to aid in determining the subject's health condition. For example, a subject with a high body temperature and certain gait characteristics in the same time frame may be more likely to be experiencing heat stroke. In another example, a subject with a high body temperature and certain vocal characteristics may be likely to have a contagious disease. Accordingly, correlations between physiological biomarkers, such as time correlations, may increase the predictive strength of the physiological biomarkers for predicting health status of a subject.

Biomarker collection systems described herein may generally include a plurality of sensors (e.g., cameras, microphones, thermometers, and the like) for contemporaneous collection of various types of biomarker-related, which may be utilized to generate physiological biomarkers predictive of health status and/or disease state. The biomarker-related data is generally collected in a standardized and time-alignable manner, which allows for more accurate physiological biomarkers to be derived from the collected biomarker-related data. For example, the collected biomarker-related and/or the physiological biomarkers may be correlated using timestamps (or otherwise time aligned) to allow more accurate biomarkers to be derived from the time aligned biomarker-related data, increasing the predictive value of the physiological biomarkers in determining various health conditions. Such systems may be deployed to screen for various health conditions in near real-time in locations outside of a medical setting. For example, such systems may be utilized at sporting events to screen participants for heat exhaustion such that those participants showing signs of heat exhaustion according to the collected biomarker-related data can be treated more promptly. In various examples, the systems may be utilized in other locations to screen for contagious diseases. For example, subjects may briefly interact with a multimodal data collection unit in the form of a kiosk before entering an event or public location to screen for signs of one or more contagious diseases.

In addition to predicting health status, the systems and methods herein may utilize collected biomarker-related data to identify other physiological biomarkers and/or correlations between physiological biomarkers associated with various health conditions. For example, a multimodal biomarker analysis system may be deployed in a healthcare setting, in-home, or may be otherwise made available to participants in clinical trials. The multimodal biomarker analysis system may collect various biomarker-related data from the participants and derive various physiological biomarkers from the biomarker-related data. The generated physiological biomarkers may be correlated with one another and/or otherwise associated with various health statuses of the participants. Various machine learning models may utilize such data to identify physiological biomarkers which are predictive of various health conditions. For example, physiological biomarkers generated based on data collected from patients with a particular flu strain may show a strong correlation between certain vocal biomarkers and body temperature biomarkers and diagnosis of the flu strain. In some examples, the identified correlations may be used to train models for diagnosis of the particular flu strain, and may be deployed for use with the multimodal data collection units described herein.

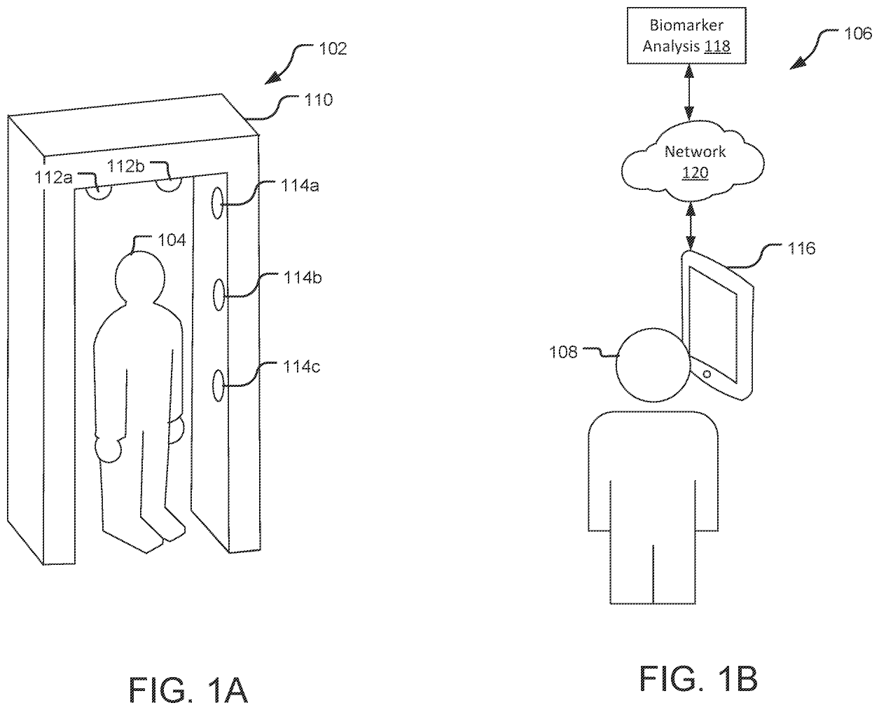

FIG. 1A illustrates an example multimodal biomarker analysis system 102 in use by a subject 104. As shown, the multimodal biomarker analysis system 102 may be configured with multiple sensors to collect numerous types of biomarker-related data from a subject at one time. Further, the data collected may be non-invasive data, allowing for relatively quick throughput of multiple subject through the biomarker analysis system 102. In some examples, various biomarker-related data may be collected and analyzed passively as a subject walks through the biomarker analysis system 102.

The multimodal biomarker analysis system 102 generally includes a multimodal data collection unit 110 including any number of sensors for collecting biomarker-related data from a subject 104. The multimodal data collection unit 110 is generally capable of collecting multiple types of biomarker-related data concurrently and in a time-efficient manner. For example, the subject 104 may walk through or stand within the multimodal data collection unit while such data is collected. For example, while the subject 104 approaches the multimodal data collection unit 110, cameras of the multimodal data collection unit 110 (e.g., cameras 112a and 112b) may be activated to capture video of the subject 104 walking, which may be utilized to generate various physiological biomarkers related to the gait of the subject 104. As the subject 104 moves closer to the multimodal data collection unit 110, additional cameras (e.g., cameras 114a-114c) may be activated to obtain video of the subject's face, which may be utilized to generate physiological biomarkers such as pupil dilation measurements, blinking rate, and the like. As the subject 104 moves through or is stationary within the multimodal data collection unit 110, the multimodal data collection unit 110 may collect additional biomarker-related data through various sensors, such as touch input, infrared video, and the like. Such biomarker-related data may be timestamped upon collection, and utilized to determine one or more health statuses or conditions of the subject.

It should be noted that the sensors may be configured to collect multiple types of information, e.g., a camera can be positioned at an elevated position with respect to the subject 104 (e.g., on the top of the data collection unit 110 or in another location, such as in a ceiling of a room including the data collection unit 110) that enables data capture sufficient to analyze a gait of the subject 104 as the subject walks or runs through or around the data collection unit 100. Also, it should be noted that the data collection unit 110 may include onboard sensors that are positioned on the unit itself as well as be in communication with off-board sensors that may be positioned in other locations (e.g., within the environment) and that the data analyzed by the data collection unit 100 may include both onboard and off-board sensor data.

In some examples, the multimodal data collection unit 110 may further include various output devices, which may be used in conjunction with sensors to capture biomarker-related data. For example, the multimodal data collection unit 110 may include a display screen, scent delivery systems, and the like. A display screen may display output requesting interaction or input from the user. For example, the display screen may display a series of questions to be answered by the subject. When the subject answers the questions, microphones of the multimodal data collection unit 110 may collect audio data which may be utilized to generate biomarkers for, for example, vocal characteristics, speech patterns, cognitive status, and the like. In some examples, the display screen may be a touch screen utilized to receive additional input from the subject. For example, a touch screen may be used to collect input to determine reaction time, assess cognitive status, and the like. In one example, the multimodal data collection unit 110 may include a scent delivery system, which may generate various identifiable scents. A touch screen may be utilized to obtain user input identifying the scents, which may be used to determine whether the user's sense of smell is in-tact.

In various examples, the various sensors of the multimodal data collection unit 110 may communicate with one another to improve the biomarker-related data collected by the sensors. For example, video data may show that a subject should move closer to a microphone in order to capture quality audio data. The subject may then be directed (e.g., by the output devices) to move closer to the microphone. In other examples, the output devices may instruct a subject to perform other actions, such as moving closer to a camera, holding a certain position for a certain amount of time, and the like. Such cross-validation between the various sensors of the multimodal data collection unit 110 leads to more standardized data collection and may, in some examples, be utilized to add additional tags to collected data. For example, captured audio data could be tagged with the subject's distance from the microphone, where the distance from the microphone is determined using video data of the subject. Such tagging could refine the system by determining parameters for improved data collection. For example, where the captured audio data is tagged with a distance of the subject from the microphone, such tagging may facilitate determination of an ideal distance of the subject from the microphone.

The multimodal data collection unit 110 may further include, or communicate with, a biomarker analysis system, which may generally utilize the collected biomarker-related data to predict or determine a health state of a subject. For example, the collected biomarker-related data may be provided to a biomarker analysis system, and the biomarker analysis system may derive physiological biomarkers from the biomarker-related data, correlate the physiological biomarkers, and utilize the physiological biomarkers to predict or determine a health status of a subject. In some examples, the biomarker analysis system may further store generated physiological biomarkers and/or use such data to train additional models or determine physiological biomarkers correlated to various health conditions or outcomes. The biomarker analysis system may, in various examples, be provided locally to the multimodal data collection unit 110 or may be accessible by the multimodal data collection unit 110 via a network or other connection.

FIG. 1B illustrates another example multimodal biomarker analysis system 106 in use by a subject 108. The multimodal biomarker analysis system 106 includes a multimodal data collection unit 116 in communication with biomarker analysis 118 via a network 120. The multimodal data collection unit 116 may be a common computing device, such as a tablet computer and may, therefore be deployed within a subject's home, a physician or other medical provider's office, a retail location, and the like. In some examples, biomarker-related data may be collected from the user using various input sensors and devices of the computing device, such as built in cameras, microphones, touch screens, and the like. In some examples, the computing device may utilize additional sensors, such as additional cameras, infrared imaging devices, and/or additional microphones to collect biomarker-related data. Such additional sensors may be accessed by the computing device via a wireless or wired communications protocol. In some examples, the multimodal data collection unit 116 may further be in communication with additional sensors and/or devices containing sensors used to collect biomarker-related data. For example, the multimodal data collection unit 116 may be a tablet computer and may communicate (e.g., via Wifi, Bluetooth, or other wired or wireless communication protocol) with a smart watch or other device including a heart rate monitor.

Like the multimodal data collection unit 110, the multimodal data collection unit 116 may generally collect timestamped biomarker-related data using various sensors, and such biomarker-related data may be provided to biomarker analysis 118 for further analysis, such as generation of physiological biomarkers, determination of health status using the physiological biomarkers and/or identifying combinations of physiological biomarkers associated with various health statuses, health conditions, and/or disease states.

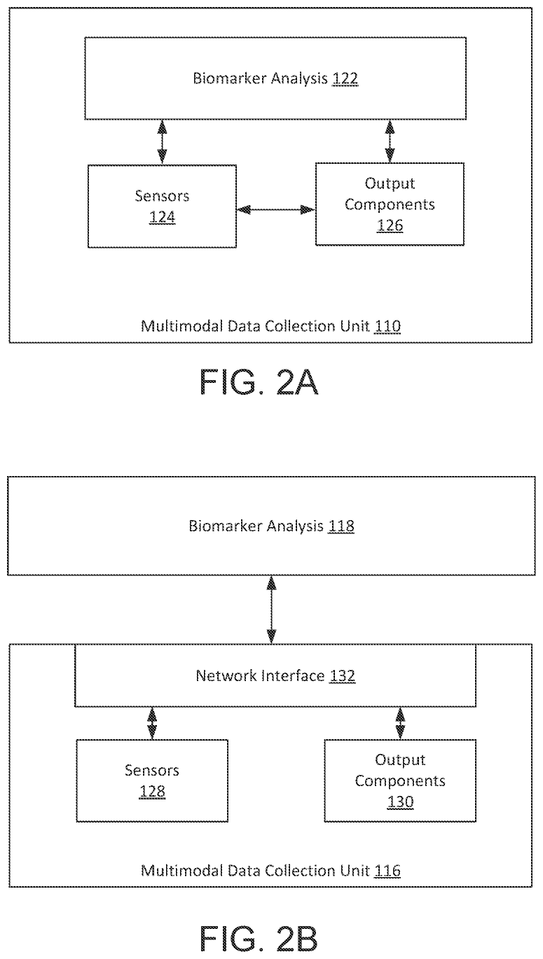

FIG. 2A illustrates a schematic diagram of an example multimodal data collection unit 110. The multimodal data collection unit 110 generally includes sensors 124, output components 126, and biomarker analysis 122. The multimodal r data collection unit 110 may include various types of sensors, such as cameras, microphones, infrared sensors, touch interfaces, buttons, and the like. In some examples, the multimodal data collection unit 110 may include multiple sensors of one type, where each sensor may be physically located or otherwise optimized to detect specific information. For example, the multimodal data collection unit 110 may include a first camera placed and configured to capture video of a subject's gait as the subject moves toward the multimodal data collection unit 110. A second camera may be placed to capture video of a subject's face to obtain various physiological biomarkers such as pupil dilation, blinking rate, and the like. In some examples, the second camera may be movable such that a height of the camera may be adjusted to capture the face of the subject. In some examples, multiple cameras of the second type may be provided in the multimodal data collection unit 110 and a different one of the cameras may be utilized to collect the biomarker-related data depending on the height of the subject.

In some examples, the sensors 124 may further include sensors configured to collect environmental data in addition to biomarker-related data from the subject. For example, humidity sensors, thermometers, and/or other sensors may collect data which may be utilized in conjunction with biomarker-related data to determine a health status of a subject. For example, a subject with some physiological biomarkers associated with heat stress may be less likely to be experiencing heat stress where environmental sensors show a low humidity and low temperature. Conversely, heat stress may be more likely where the environmental sensors show high temperature and high humidity. In various examples, the multimodal data collection unit 110 may include additional components or functionality for collecting additional information (e.g., context input) that may be utilized in conjunction with biomarker-related data to determine a health status of a subject. For example, a multimodal data collection unit 110 may be placed in a home of a subject, and may collect biomarker-related video data to track medication compliance. The multimodal data collection unit 110 may include a touch screen configured to display an electric form for the user to record the time medication is taken. Such information may be utilized to enhance accuracy of the biomarker-related video data in determining medication compliance.

In another example, the multimodal data collection unit 110 may collect other types of context input from other types of sensors, such as wearable or other sensors in communication with the multimodal data collection unit 110. For example, cameras of the multimodal data collection unit 110 may detect exertion or metabolic rate. A wearable sensor in communication with the multimodal data collection unit 110 may be utilized to detect heart rate and the like during collection of the video data. The information from the wearable sensor (e.g., the exertion rate during heavy exercise) could influence how the physiological biomarkers are interpreted and correlated with a health status of the subject.

Context data may, in various examples, be utilized to tag other types of data. For example, audio data collected by the multimodal data collection unit 110 may be tagged using temperature and humidity data collected by environmental sensors of the multimodal data collection unit 110. In addition to identifying additional correlations, such tagged data may provide an understanding of whether certain environmental conditions are confounding variables. Information about confounding variables may be useful, for example, in clinical trials.

Output components 126 may include, for example, touchscreen or other display interfaces, lights, scent delivery systems, audio output devices (e.g., speakers), and the like. Such output components 126 may be utilized to guide subjects through the data collection process and/or to provide output utilized in conjunction with the sensors 124 to collect biomarker-related data. For example, a display may prompt the user to answer specific questions so that the sensors 124 can collect audio data. Similarly, users may be instructed to provide some input (e.g., to a touchscreen interface) related to output provided by the output components 126 such as audio output, scents, light output, and the like. Such user provided input may be utilized to generate physiological biomarkers related to vision, sense of smell, hearing, and the like. In some examples, prompts may be dynamic. That is, prompts may be presented to collect additional information in response to positive indications of specific physiological biomarkers. For example, if collected audio data indicates a potential viral infection, additional prompts could be provided to a subject to collect information about additional symptoms such as, muscle ache, headache, and the like. Such additional information collected from these prompts may improve the probability of detection of the viral infection.

Biomarker analysis 122 may be implemented by various computing resources of the multimodal data collection unit 110 and may generally include functionality for analyzing the collected biomarker-related data to determine health status of a subject and/or to identify physiological biomarkers correlated with health status of a subject. In some examples, biomarker analysis 122 may be provided with or instructed to use a particular protocol for determination of a particular health state or status based on the intended use of the multimodal data collection unit 110. For example, where the multimodal data collection unit 110 is deployed at an athletic event, biomarker analysis 122 may be instructed to use a protocol for predicting heat stress. Where the multimodal data collection unit 110 is deployed to detect contagious disease, biomarker analysis 122 may be configured to use a different protocol for detecting a contagious disease.

Generally, a particular protocol may include a machine learning model which determines or predicts a relevant health state based on various physiological biomarkers. A protocol may further include instructions for calibrating sensors, obtaining various types of biomarker-related data (e.g., prompting subjects to answer questions to obtain audio data), and for determining particular physiological biomarkers from the collected biomarker-related data. In various examples, such machine learning models may employ and/or utilize various privacy preserving approaches such as federated learning and the like.

FIG. 2B illustrates a schematic diagram of an additional multimodal data collection unit 116. The multimodal data collection unit 116 generally includes sensors 128, output components 130, and a network interface 132. The network interface 132 may generally be utilized to communicate with biomarker analysis 118. For example, biomarker-related data may be collected by the sensors 128 of the multimodal data collection unit 116, and such biomarker-related data may be communicated to biomarker analysis 118 for determination of a health status.

While FIGS. 2A and 2B show a biomarker analysis system being either fully incorporated into, or fully remote from, a multimodal data collection unit, in various examples, some functionality of a biomarker analysis system may be local to a multimodal data collection unit while some functionality of the biomarker analysis system may be remote from the multimodal data collection unit.

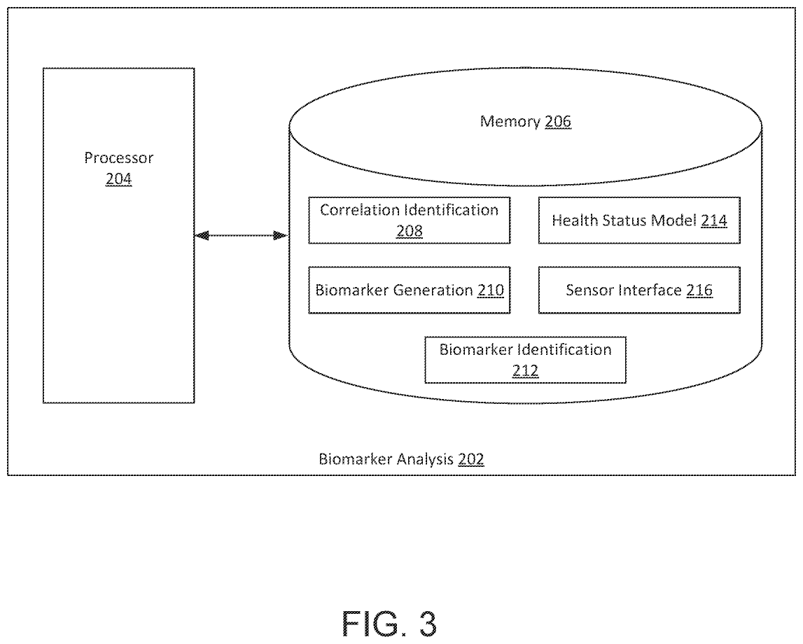

FIG. 3 illustrates a schematic diagram of an example biomarker analysis system 202. The biomarker analysis system 202 may be used to implement, or may be implemented by, biomarker analysis 122 and/or biomarker analysis 118. In some examples, the biomarker analysis system 202 may be located within a multimodal data collection unit. In other examples, some or all of the components of the biomarker analysis system 202 may be remote from a multimodal data collection unit. For example, the biomarker analysis system 202 (and/or some portions of the biomarker analysis system 202) may be provided as a cloud hosted service and a multimodal data collection unit may provide collected biomarker-related data to the biomarker analysis system 202 and/or receive various output from the biomarker analysis system 202.

In various examples, the biomarker analysis system 202 may include or utilize one or more hosts or combinations of compute resources which may be located, for example, at one or more servers, cloud computing platforms, computing clusters, and the like. Generally, the biomarker analysis system 202 is implemented by compute resources including hardware for memory 206 and one or more processors 204. For example, the biomarker analysis system 202 may utilize or include one or more processors, such as a CPU, GPU, and/or programmable or configurable logic.

In some embodiments, various components of the biomarker analysis system 202 may be distributed across various computing resources, such that the components of the biomarker analysis system 202 communicate with one another through a network and/or other communications protocols. For example, in some embodiments, the biomarker analysis system 202 may be implemented as a serverless service, where computing resources for various components of the biomarker analysis system 202 may be located across various computing environments (e.g., cloud platforms) and may be reallocated dynamically and/or automatically according to, for example, resource usage of the biomarker analysis system 202. In various implementations, the biomarker analysis system 202 may be implemented using organizational processing constructs such as functions implemented by worker elements allocated with compute resources, containers, virtual machines, and the like.

The memory 206 may include instructions for various functions of the biomarker analysis system 202 which, when executed by the processor 204, perform various functions of the biomarker analysis system 202. The memory 206 may further store data and/or instructions for retrieving data used by the biomarker analysis system 202. Similar to the processor 204, memory resources utilized by the biomarker analysis system 202 may be distributed across various physical computing devices. In some examples, memory 206 may access instructions and/or data from other devices or locations, and such instructions and/or data may be read into memory 206 to implement the biomarker analysis system 202.

In various embodiments, the memory 206 may include instructions for a sensor interface 216. The sensor interface 216 may generally receive biomarker-related data from various sensors of a multimodal data collection unit. The sensor interface 216 may, in some examples, be configured to time-stamp biomarker-related data as it is received from various sensors. Such timestamps may be utilized by other components of biomarker analysis 202 to time align the biomarker-related data, with the time aligned and standardized biomarker-related data providing a robust data set for derivation of physiological biomarkers.

In various embodiments, memory 206 may include instructions for biomarker generation 210. Biomarker generation 210 may generally receive biomarker-related data (e.g., time aligned multimodal biomarker-related data) from sensors of a multimodal data collection unit and may process the biomarker-related data to generate one or more physiological biomarkers. In some examples, a physiological biomarker may be generated based on one type of biomarker-related data. In some examples, biomarker generation may generate particular biomarkers based on a particular protocol being employed (e.g., a particular health state being predicted). In some examples, where no particular protocol is used or where biomarker-related data is being collected for identification of physiological biomarkers associated with a health status, biomarker generation 210 may generate as many physiological biomarkers as possible from the provided biomarker-related data. Biomarker generation 210 may generally associate physiological biomarkers with some time measurement, such as a timestamp obtained from the biomarker-related data.

Memory 206 may further include instructions for correlation identification 208. Correlation identification may, in some examples, time align the various biomarker-related data received from a multimodal data collection unit. Such time alignment may include reformatting and/or standardizing timestamps or other time measures associated with the biomarker-related data to place the biomarker-related data on a common time scale. When on a common time scale, correlation identification 208 may identify time correlations between the physiological biomarkers, such as those that occur sequentially (and in what order), concurrently, and/or simultaneously. Such correlations may be, along with the physiological biomarkers themselves, predictive of a health status. For example, a vocal disruption followed approximately two minutes later by a walking gait disruption may be shown to be a strong predictor of a certain health condition or health status, and time alignment may assist in identifying and utilizing such correlations for determination of health status.

In various examples, the memory 206 further includes a health status model 214. The health status model may be a machine learning model or other algorithm configured to determine or predict a particular health status or set of health statuses based on physiological biomarkers collected from a subjects. For example, biomarker generation 210 may provide the generated biomarkers to the health status model 214 The health status model 214 may then produce output providing a health status of the subject. Such output may, in some examples, include a binary classification, along with some confidence metric for the classification. For example, the health status model 214 may state that the subject is likely suffering from heat stress, with a 75% probability. Such output may be provided to the multimodal data collection unit and/or another designated computing device for relaying the results to the subject and/or another user. For example, the multimodal data collection unit may include a display configured to display health status results. Alternatively or additionally, an additional user device (e.g., a user device belonging to a supervisor or other user overseeing use of the multimodal data collection unit) may be in communication with the multimodal data collection unit and/or the biomarker analysis system 202 and may be provided with the output generated by the health status model 214.

In various examples, the memory 206 may further include instructions for biomarker identification 212. Biomarker identification 212 may generally utilize biomarkers tagged with known health statuses to identify particular biomarkers and correlations between such biomarkers which may be highly predictive of such health statuses. In some examples, physiological biomarkers (e.g., those generated by biomarker generation 210) and/or time aligned biomarker-related data may be associated with a particular subject and stored (either locally to or remote from biomarker analysis 202) for later use by biomarker identification. A user may provide the biomarker analysis system 202 with information about the health status of the subject at the time of collection (e.g., presence of a particular disease as corroborated by test results, a separate diagnosis, and the like). The stored physiological biomarkers may then be tagged or otherwise associated with the received health status, and may be provided to biomarker identification 212. Biomarker identification 212 may then utilize the tagged biomarkers, along with tagged biomarkers obtained from other subjects, to identify physiological biomarkers and correlations between the physiological biomarkers associated with various health statuses. For example, biomarker identification may include one or more neural networks or other deep learning models which may identify patterns in the tagged data.

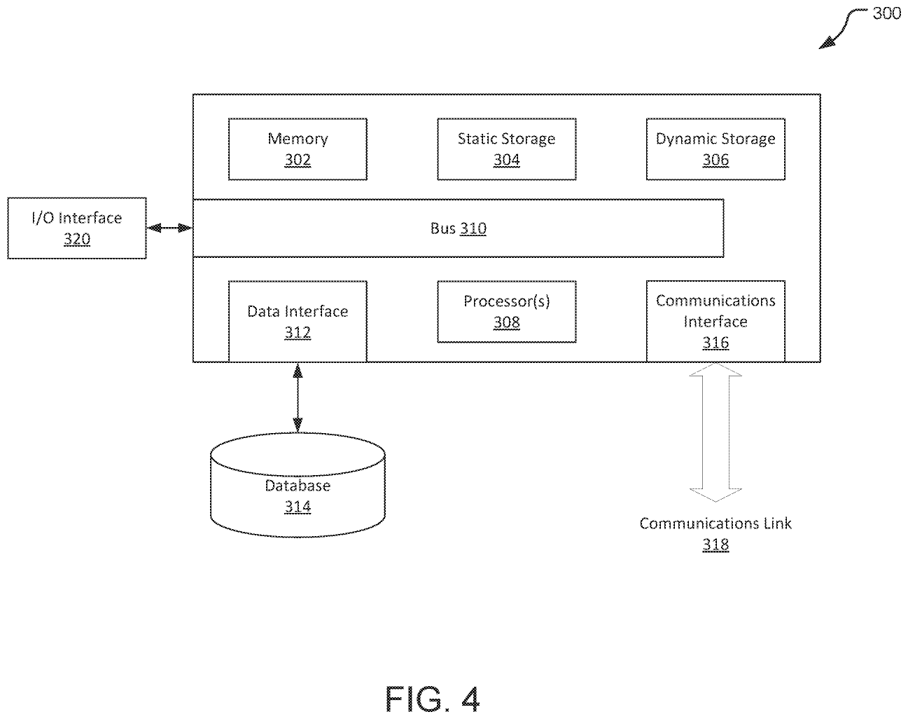

FIG. 4 illustrates an example computing system 300 that may be used for implementing various embodiments in the examples described herein. For example, in various embodiments, components of the biomarker analysis system 102 and/or the biomarker analysis system 106 may be implemented by one or several computing systems 300. For example, the multimodal data collection unit 110, the multimodal data collection unit 116, biomarker analysis 118, and/or biomarker analysis 122 may include or be implemented by a computing system 300. This disclosure contemplates any suitable number of computing systems 300. For example, the computing system 300 may be a server, a desktop computing system, a mainframe, a mesh of computing systems, a laptop or notebook computing system, a tablet computing system, an embedded computer system, a system-on-chip, a single-board computing system, or a combination of two or more of these. Where appropriate, the computing system 300 may include one or more computing systems; be unitary or distributed; span multiple locations; span multiple machines; span multiple data centers; or reside in a cloud, which may include one or more cloud components in one or more networks.

Computing system 300 includes a bus 310 (e.g., an address bus and a data bus) or other communication mechanism for communicating information, which interconnects subsystems and devices, such as processor 308, memory 302 (e.g., RAM), static storage 304 (e.g., ROM), dynamic storage 306 (e.g., magnetic or optical), communications interface 316 (e.g., modem, Ethernet card, a network interface controller (NIC) or network adapter for communicating with an Ethernet or other wire-based network, a wireless NIC (WNIC) or wireless adapter for communicating with a wireless network, such as a WI-FI network), input/output (I/O) interface 320 (e.g., keyboard, keypad, mouse, microphone). In particular embodiments, the computing system 300 may include one or more of any such components.

In particular embodiments, processor 308 includes hardware for executing instructions, such as those making up a computer program. For example, a processor 308 may execute instructions for various components of a biomarker analysis system. The processor 308 circuitry includes circuitry for performing various processing functions, such as executing specific software for performing specific calculations or tasks. In particular embodiments, I/O interface 320 includes hardware, software, or both, providing one or more interfaces for communication between computing system 300 and one or more I/O devices. Computing system 300 may include one or more of these I/O devices, where appropriate. One or more of these I/O devices may enable communication between a person and computing system 300.

In particular embodiments, the communications interface 316 includes hardware, software, or both providing one or more interfaces for communication (such as, for example, packet-based communication) between computing system 300 and one or more other computer systems or one or more networks. One or more memory buses (which may each include an address bus and a data bus) may couple processor 308 to memory 302. Bus 310 may include one or more memory buses, as described below. In particular embodiments, one or more memory management units (MMUs) reside between processor 308 and memory 302 and facilitate accesses to memory 302 requested by processor 308. In particular embodiments, bus 310 includes hardware, software, or both coupling components of computing system 300 to each other.

According to particular embodiments, computing system 300 performs specific operations by processor 308 executing one or more sequences of one or more instructions contained in memory 302. For example, instructions for various components of the biomarker analysis system 202 may be contained in memory 302 and may be executed by the processor 308. Such instructions may be read into memory 302 from another computer readable/usable medium, such as static storage 304 or dynamic storage 306. In alternative embodiments, hard-wired circuitry may be used in place of or in combination with software instructions. Thus, particular embodiments are not limited to any specific combination of hardware circuitry and/or software. In various embodiments, the term “logic” means any combination of software or hardware that is used to implement all or part of particular embodiments disclosed herein.

The term “computer readable medium” or “computer usable medium” as used herein refers to any medium that participates in providing instructions to processor 308 for execution. Such a medium may take many forms, including but not limited to, nonvolatile media and volatile media. Non-volatile media includes, for example, optical or magnetic disks, such as static storage 304 or dynamic storage 306. Volatile media includes dynamic memory, such as memory 302.

Computing system 300 may transmit and receive messages, data, and instructions, including program, e.g., application code, through communications link 318 and communications interface 316. Received program code may be executed by processor 308 as it is received, and/or stored in static storage 304 or dynamic storage 306, or other storage for later execution. A database 314 may be used to store data accessible by the computing system 300 by way of data interface 312. For example, projection settings and predetermined positions of ride vehicles may be stored using a database 314. In various examples, a communications link 318 at the multimodal data collection unit 116 may communicate with biomarker analysis 118, and/or computing components within the network 120.

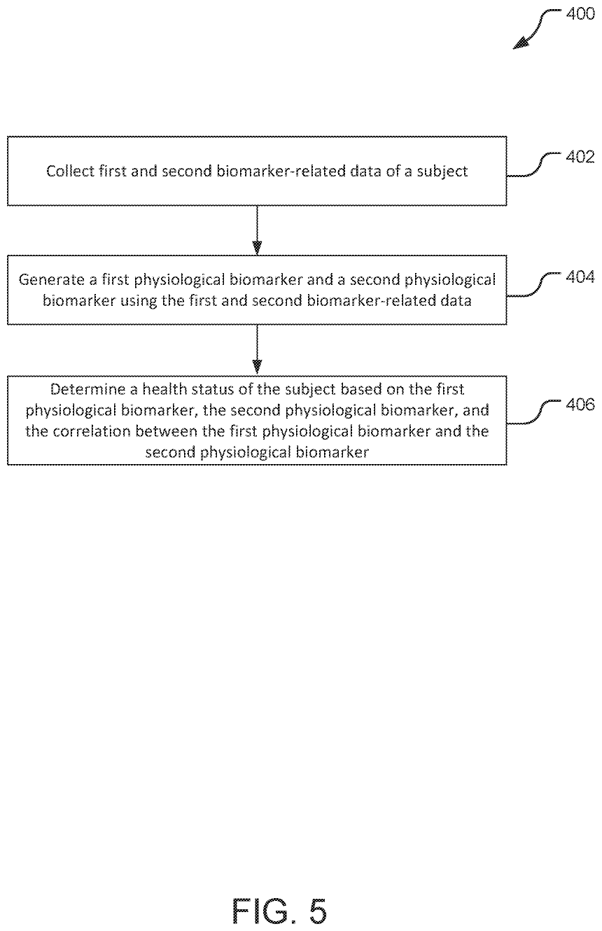

FIG. 5 illustrates an example process 400 for determining health status of a subject using correlations between physiological biomarkers. At block 402, the multimodal biomarker analysis system collects first and second biomarker-related data of a subject. For example, a multimodal data collection unit 110 may collect biomarker-related data from a subject 104 interacting with the multimodal data collection unit 110. In some examples, the multimodal data collection unit 110 may be configured to execute a specific protocol to collect the biomarker-related data. That is, the multimodal data collection unit 110 may calibrate various sensors to collect biomarker-related data relevant to a particular health status being screened for. In accordance with a particular protocol, the multimodal data collection unit 110 may further generate specific output (e.g., instructions, sensory output, and/or interactive output) to facilitate collection of the desired biomarker-related data. For example, the multimodal data collection unit 110 may be instructed to implement a protocol to detect heat stress and may, accordingly, collect video biomarker-related data which may be used to generate gait biomarkers of a subject and audio biomarker-related data which may be used to generate vocal biomarkers of the subject.

The multimodal data collection unit 110 generally collects biomarker-related data using sensors integrated into and/or in communication with the multimodal data collection unit 110. For example, the multimodal data collection unit 110 may activate a camera focused on the subject while the subject walks towards or through the multimodal data collection unit 110. In some examples, the multimodal data collection unit 110 may provide some output to elicit certain actions from the subject for the collection of biomarker-related data. For example, a display of the multimodal data collection unit 110 may instruct a user to walk towards or through the multimodal data collection unit 110. As the user walks, a camera of the multimodal data collection unit may collect video showing the user's gait. Similarly, the display may display prompts and/or audio prompts may be provided to a user to elicit vocal output, which may be collected as audio biomarker-related data.

In some examples, the output may be configured such that the same biomarker-related data may be utilized to generate more than one physiological biomarker. For example, a user may be prompted to orally answer questions provided by the multimodal data collection unit 110. The audio biomarker-related data may be analyzed to determine the answers to such questions, which may be used to generate physiological biomarkers related to a cognitive status of the subject. The same audio biomarker-related data may be utilized to generate vocal biomarkers measuring vocal characteristics such as hoarseness of the subject. In this way, the multimodal data collection unit allows for predictions related to various biomarkers to be formed in a rapid high-throughput manner. For example, by collecting walking gait as a subject approaches an area with sensors collecting vocal recordings and/or input through a touch interface, and collecting the subject's answers to cognitive tests while collecting audio and facial data from the subject allows for all collected types of data to be correlated to one another for use in training of machine learning models, analysis, and the like.

Biomarker-related data collected by the multimodal data collection unit 110 may generally be timestamped and provided to a biomarker analysis system (e.g., the biomarker analysis system 202) which may be partially or fully integrated into, or remote from, the multimodal data collection unit 110. For example, the biomarker-related data may be provided to a sensor interface 216 of the biomarker analysis system 202.

In some examples biomarker-related data collected by the multimodal data collection unit 110 may be anonymized or otherwise secured to protect privacy of the subjects. For example, biomarker-related data may be stored on a blockchain or analyzed using secured federated analysis techniques. Subject data may further be depersonalized or otherwise modified to obfuscate any identifiers of the subject. For example, homomorphic encryption, zero-knowledge proofs, or other methods may be used to depersonalize or de-identify the data, access to the data, and/or other operations on the data.

The multimodal biomarker system generates a first physiological biomarker and a second physiological biomarker using the first and second biomarker-related data at block 404. For example, the sensor interface 216 may provide the biomarker-related data to biomarker generation 210. Biomarker generation 210 may analyze the biomarker-related data to generate selected biomarkers. In some examples, one type of biomarker-related data may be utilized to generate one physiological biomarker. For example, biomarker generation 210 may generate a body temperature physiological biomarker based thermal imaging biomarker-related data. In some examples, multiple physiological biomarkers may be generated from one type of biomarker-related data. For example, video data of a subject's face may be utilized to generate physiological biomarkers for a blinking rate of the subject and pupil dilation of the subject. In some examples, multiple types of biomarker-related data may be combined to generate one type of physiological biomarker. Generally, biomarker generation 210 may correlate any generated physiological biomarkers with relevant timestamps or other time data, which may be derived from the biomarker-related data.

In various examples, the specific biomarkers generated at block 404 may be determined based on a training deployment of the multimodal data collection unit 110. For example, biomarker-related data may be collected by the various sensors of the multimodal data collection unit 110 in a training deployment. A model may be trained using the biomarker-related data to identify various physiological biomarkers that may be generated based on the types of biomarker-related data collected by the multimodal data collection unit 110. Such identified biomarkers may be the physiological biomarkers generated in a deployment scenario (e.g., where the multimodal biomarker system is used to determine a health status of a subject).

The multimodal biomarker system determines a health status of the subject at block 406. The health status of the subject is based on the first physiological biomarker, the second physiological biomarker, and the correlation between the first physiological biomarker and the second physiological biomarker. The generated physiological biomarkers and the correlations between the physiological biomarkers may be provided to a health status model 214 to determine a health status of a subject. The health status model 214 may generally be trained to generate output related to a health status (e.g., a binary determination of whether a particular state or disease is likely present) based on physiological biomarkers and the correlations (e.g., timing correlations) between the physiological biomarkers. In some examples, the health status model 214 may generate such output for a number of health statuses at the same time. That is the health status model 214 may be configured to detect multiple health conditions and/or diseases from a set of physiological biomarkers and correlations between the physiological biomarkers in the set.

In various examples, output generated by the health status model 214 may be provided to an output device of the multimodal data collection unit 110. For example, the health status model 214 may generate a binary output indicating whether the subject likely has a communicable disease, along with, in some examples, a probability of the subject having the communicable disease. The binary output may be provided to a display of the multimodal data collection unit 110 to alert the subject and/or an operator of the multimodal data collection unit 110 of the result. In some examples, such output may be provided, additionally or alternatively, to a user device

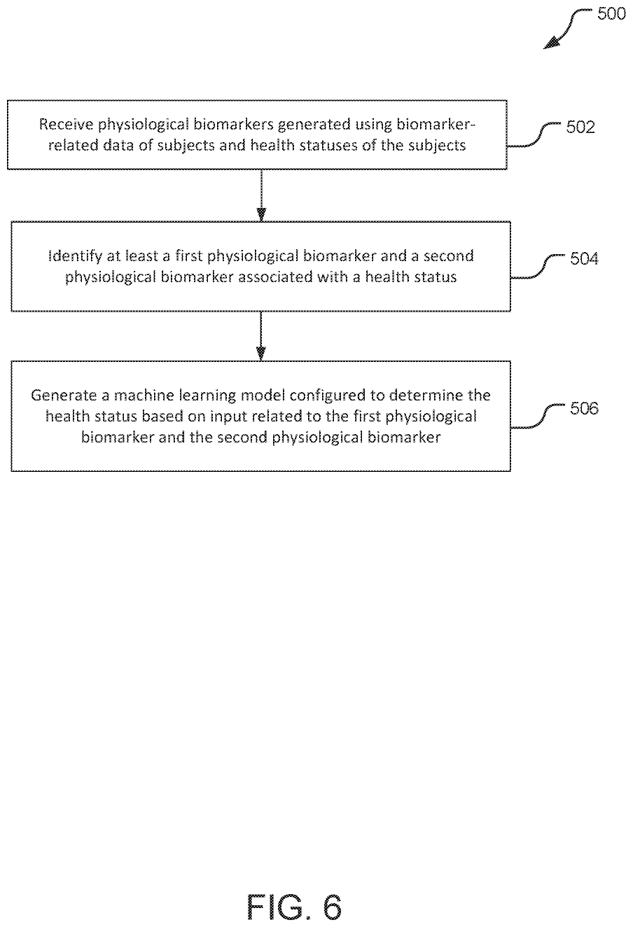

FIG. 6 illustrates an example process 500 for generating machine learning models for detecting health status using physiological biomarkers and cross-correlations of physiological biomarkers. For example, a cross-correlation between two physiological biomarkers may be an indication that both physiological biomarkers and/or the combination of both physiological biomarkers, are predictive of a particular health status. At block 502, the multimodal biomarker system receives physiological biomarkers generated using biomarker-related data of subjects and health statuses of the subjects. In some examples, the multimodal biomarker system may receive the biomarker-related data over time, and such data may be tagged to individual subjects. For example, participants in a clinical trial, regular patients at a medical provider, individual users within their home, or others regularly interacting with the multimodal biomarker system to measure biomarker-related data. The system may further be provided with known health statuses or disease outcomes for individual subjects at particular times, and the relevant biomarker-related data may be tagged with such outcomes. In these examples, the process 500 may identify relevant physiological biomarkers and/or combinations of physiological biomarkers that may be highly predictive of the known health statuses, diseases, and/or physical conditions, allowing for the generation and training of health status models for use in detecting such health statuses, diseases, and/or conditions using the multimodal biomarker system.

In some examples, the multimodal biomarker system may retain biomarker-related data already used by the multimodal biomarker system in predicting health statuses, diseases, and/or physical conditions. The multimodal biomarker system may generate additional biomarkers from the biomarker-related data to identify new physiological biomarkers which may also be predictive of the health statuses, diseases, and/or physical conditions. The multimodal biomarker system may further identify new correlations or relationships between physiological biomarkers which are highly predictive of health statuses, diseases, and/or physical conditions. In some examples, such new physiological biomarkers and/or correlations between physiological biomarkers may be used to improve upon or replace existing health status models within biomarker analysis systems (e.g., health status model 214 of biomarker analysis 202). In such examples, biomarker-related data may be tagged with output from an existing health status model 214 for later use by biomarker identification 212.

Biomarker generation 210 may generate physiological biomarkers using the stored and tagged biomarker-related data. In some examples, previously generated physiological biomarkers (e.g., those generated during the process 400 may be stored as tagged data and biomarker generation 210 may generate new physiological biomarkers from stored biomarker-related data. The physiological biomarkers may retain any timestamps or other collection time tags, and may retain any outcome or health status tags associated with the received and/or stored biomarker-related data. The tagged physiological biomarkers may then be provided to biomarker identification 212 as, for example, tagged training data.

The multimodal biomarker system identifies, at block 504, at least a first physiological biomarker and a second physiological biomarker associated with a health status. For example, biomarker identification 212 may receive the tagged data and may identify relationships between various health statuses and the physiological biomarkers. Biomarker identification may, in some examples, be a neural network or other unsupervised learning model which uses the training data to identify latent relationships between the various physiological biomarkers, correlations between the physiological biomarkers, and health statuses.

At block 506, the multimodal biomarker system generates a machine learning model configured to determine the health status based on input related to the first physiological biomarker and the second physiological biomarker. In various examples, such a model may then be deployed within the biomarker analysis system 202 as a health status model 214 configured to determine or predict a particular health status.

Using the method 500, use of a multimodal biomarker analysis system may be extended to additional health statuses, diseases, and/or physical conditions as physiological biomarkers are identified which are predictive of such health statuses, diseases, and/or physical conditions, which may provide improved identification and/or diagnoses. Such improved identification and diagnosis may be especially helpful where the physiological biomarkers are found to be predictive of communicable diseases, as people having the communicable diseases may be more easily identified and containment measures may be put in place to prevent the spread of such diseases. Additionally, where a communicable disease (e.g., a virus) mutates rapidly and such mutations cause different symptoms in those infected, the method 500 may be able to adapt to the mutations more quickly and identify new biomarkers that may be predictive of such mutations.

The multimodal biomarker analysis system described herein may be adaptable and/or updatable by generating and/or swapping out various machine learning models. Transfer learning may, in such examples, be utilized to train a new model from an existing model. For example, a model used to detect a respiratory virus using vocal data may be created using transfer learning techniques on machine learning models generated for voice-based dementia detection. In this manner, the multimodal data collected by the multimodal biomarker analysis system may be utilized to identify new biomarker correlations with various health conditions that may have been unknown at the time of collection, further increasing the adaptability of the multimodal biomarker analysis system.

The description of certain embodiments included herein is merely exemplary in nature and is in no way intended to limit the scope of the disclosure or its applications or uses. In the included detailed description of embodiments of the present systems and methods, reference is made to the accompanying drawings which form a part hereof, and which are shown by way of illustration specific to embodiments in which the described systems and methods may be practiced. These embodiments are described in sufficient detail to enable those skilled in the art to practice presently disclosed systems and methods, and it is to be understood that other embodiments may be utilized, and that structural and logical changes may be made without departing from the spirit and scope of the disclosure. Moreover, for the purpose of clarity, detailed descriptions of certain features will not be discussed when they would be apparent to those with skill in the art so as not to obscure the description of embodiments of the disclosure. The included detailed description is therefore not to be taken in a limiting sense, and the scope of the disclosure is defined only by the appended claims.

From the foregoing it will be appreciated that, although specific embodiments of the invention have been described herein for purposes of illustration, various modifications may be made without deviating from the spirit and scope of the invention.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

Of course, it is to be appreciated that any one of the examples, embodiments or processes described herein may be combined with one or more other examples, embodiments and/or processes or be separated and/or performed amongst separate devices or device portions in accordance with the present systems, devices and methods.

Finally, the above discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

What is claimed is:

1. A method comprising:
collecting first biomarker-related data of a subject from a first sensor and second biomarker-related of the subject from a second sensor, the first biomarker-related data being tagged with a first time of collection of the first biomarker-related data from the first sensor, the second biomarker-related data being tagged with a second time of collection of the second biomarker-related data from the second sensor;
generating a first physiological biomarker using the first biomarker-related data and a second physiological biomarker using the second biomarker-related data; and
determining a health status of the subject based on the first physiological biomarker and the second physiological biomarker.

2. The method of claim 1, wherein the first biomarker-related data and the second biomarker-related data are non-invasive biomarker-related data.

3. The method of claim 1, wherein the health status of the subject is further determined based on at least first environmental data associated with an environment where the subject is located.

4. The method of claim 1, wherein the health status of the subject is determined using a machine learning model trained to predict the health status based on the first physiological biomarker and the second physiological biomarker.

5. The method of claim 1, wherein the first biomarker-related data is collected responsive to first output provided to the user, wherein the user interacts with the first output to produce the first biomarker-related data.

6. The method of claim 1, wherein the health status is determined further based on a third physiological biomarker, wherein the third physiological biomarker is generated using third biomarker-related data collected by a third sensor.

7. The method of claim 1, wherein the first biomarker-related data and the second biomarker-related data are collected concurrently.

8. The method of claim 1, wherein the first sensor and the second sensor are contained in a multimodal data collection unit, the multimodal data collection unit including a display configured to generate instructions for the subject to follow during the collection of the first biomarker-related data and the second biomarker-related data, wherein the display is further configured to display the health status of the subject responsive to the determination of the health status.

9. One or more non-transitory computer readable media encoded with instructions which, when executed by one or more processors, cause the one or more processors to:
receive a plurality of physiological biomarkers generated based on biomarker-related data collected from a plurality of subjects, the plurality of physiological biomarkers being associated with a plurality of health statuses of the plurality of subjects;
identify at least a first physiological biomarker of the plurality of physiological biomarkers and a second physiological biomarker of the plurality of physiological biomarkers associated with a health status of the plurality of health statuses; and
generate a machine learning model configured to determine the health status based on input related to the first physiological biomarker and the second physiological biomarker.

10. The one or more non-transitory computer readable media of claim 9, wherein the first physiological biomarker and the second physiological biomarker are associated with timestamps, wherein the correlation between the first physiological biomarker and the second physiological biomarker is a time correlation determined using the respective timestamps.

11. The one or more non-transitory computer readable media of claim 9, wherein the one or more processors are further configured to provide the machine learning model to a multimodal data collection unit, the multimodal data collection unit including sensors configured to collect the biomarker-related data and determine the health status using the machine learning model.

12. The one or more non-transitory computer readable media of claim 9, wherein the one or more processors are further configured to identify the first physiological biomarker and the second physiological biomarker using a neural network configured to identify relationships between the plurality of physiological biomarkers and the plurality of health statuses.

13. The one or more non-transitory computer readable media of claim 9, wherein the biomarker-related data is collected by a multimodal data collection unit including a plurality of sensors configured to capture the biomarker-related data, wherein the processors are further configured to provide the generated machine learning model to the multimodal data collection unit.

14. A biomarker correlation system comprising:
a plurality of sensors configured to collect biomarker-related data from a subject; and
one or more processors configured to:
receive first biomarker-related data of the subject from a first sensor of the plurality of sensors and second biomarker-related data of the subject from a second sensor of the plurality of sensors,
generate a first physiological biomarker using the first biomarker-related data and a second physiological biomarker using the second biomarker-related data, and
determine a health status of the subject based on the first physiological biomarker and the second physiological biomarker.

15. The biomarker correlation system of claim 14, wherein the first biomarker-related data and the second biomarker-related data are non-invasive biomarker-related data.

16. The biomarker correlation system of claim 14, further comprising a second plurality of sensors configured to collect environmental data from an environment where the subject is located, wherein the one or more processors are configured to determine the health status of the subject further based on the environmental data.

17. The biomarker correlation system of claim 14, wherein the one or more processors are configured to determine the health status of the subject using a machine learning model trained to predict the health status based on the first physiological biomarker and the second physiological biomarker.

18. The biomarker correlation system of claim 14, further comprising at least one output device configured to provide first output to the user, wherein the user interacts with the first output to produce the first biomarker-related data.

19. The biomarker correlation system of claim 14, further comprising a third sensor configured to collect third biomarker-related data, wherein the health status is determined further based on a third physiological biomarker generated using the third biomarker-related data.

20. The biomarker correlation system of claim 14, wherein the one or more processors are configured to collect the first biomarker-related data and the second biomarker-related data concurrently.
